(19) United States
(12) Patent Application Publication
(10) Pub. No.: US 2025/0359919 A1
(43) Pub. Date: Nov. 27, 2025

(54) TEMPERATURE CONTROL SYSTEM FOR MULTI-RADIOFREQUENCY ABLATION NEEDLES AND METHOD THEREFOR

(71) Applicant: SHANGHAI MAAGI MEDICAL TECHNOLOGY CO., LTD, Shanghai (CN)

(72) Inventors: Yifei WANG, Shanghai (CN); Ke ZOU, Shanghai (CN)

(21) Appl. No.: 19/104,633

(22) PCT Filed: Nov. 14, 2022
(86) PCT No.: PCT/CN2022/131746

§ 371 Date: Feb. 18, 2025

(30) Foreign Application Priority Data
Aug. 18, 2022 (CN) 202210993336.7

(51) Int. Cl. A61B 18/14; A61B 18/00; A61B 18/12; G16H 40/63

(52) U.S. Cl. CPC A61B 18/14; A61B 18/1206; G16H 40/63; A61B 2018/00577; A61B 2018/00642; A61B 2018/00672; A61B 2018/00678; A61B 2018/00714; A61B 2018/00767; A61B 2018/00791; A61B 2018/143

(57) ABSTRACT

A temperature control system includes N radio frequency ablation needles, a radio frequency generator, an ablation switch array, and a controller; the controller is configured to: obtain N temperature values from the temperature sensors on the N radio frequency ablation needles, calculate a dynamically rising current reference temperature based on the N temperature values, determine a current upper temperature limit and a current lower temperature limit based on that current reference temperature, control the ablation switch array such that radio frequency ablation needles with temperature values exceeding the current upper temperature limit are disconnected from the radio frequency generator, and radio frequency ablation needles with temperature values below the current lower temperature limit are connected to the radio frequency generator.

### FIELD

This application relates to radio frequency ablation, particularly to temperature control technology for multiple radio frequency ablation needles.

### BACKGROUND

This section is intended to provide background or context for understanding the embodiments of the present application, and is provided for reference only. It should not be construed as an admission by the applicant that this section constitutes prior art publicly disclosed before the filing date of this application.

Tumor thermal ablation, as a minimally invasive treatment method, has been widely applied in the treatment of liver, breast, kidney and other tumors. Radio frequency (RF) ablation, as one of these methods, works by using heat generated from high-frequency alternating current passing through the human body to cause coagulative necrosis of tumor cells, thereby achieving therapeutic purposes, and has been proven effective in the treatment of liver tumors. However, the radio frequency ablation range achievable by a single radio frequency ablation needle (also referred to as an ablation electrode or electrode needle) is limited, generally providing good therapeutic effects for tumors with diameters less than 3 cm. For larger tumor volumes, commonly used methods include single-needle multi-site overlapping ablation (i.e., using a single radio frequency ablation needle to ablate multiple times at different positions), improved electrode needles (such as umbrella needles), and multi-needle simultaneous ablation. Among these, multi-site overlapping ablation significantly prolongs treatment time and easily forms uncovered areas; improved electrode needles have limited expansion of the ablation range and are not suitable for simultaneous multi-site ablation; multi-needle simultaneous ablation has advantages such as operational flexibility and large ablation range, and can achieve good ablation effects for larger tumors.

For example, Chinese Patent (Application No. 201720054631.0) discloses a radio frequency ablation device for multi-electrode synchronous treatment. This patent performs multi-electrode synchronous ablation on a larger single tumor. During the ablation process, three high-frequency current generating modules provide energy to three radio frequency ablation needles respectively, and each high-frequency current generating module and its corresponding radio frequency ablation needle dynamically adjust according to power and temperature until the ablation treatment is completed. This solution uses multiple high-frequency current generating modules, which is costly, and different high-frequency current generating modules may interfere with each other.

Furthermore, during multi-needle ablation, due to the different biological tissue characteristics near the location of each electrode needle, temperature rise rates can be inconsistent, leading to excessive temperature differences between different radio frequency ablation needles during the heating process, thereby affecting treatment efficacy. Excessively high temperatures can cause over-burning, resulting in tissue carbonization that affects energy output, necrotic tissue that is difficult to absorb prolonging patient recovery time, severe pain, or tissue adhesion to the probe. Temperatures that are too low cannot achieve the purpose of ablating the target lesion. Therefore, precise energy delivery and control are particularly important during simultaneous multi-needle ablation.

### SUMMARY

The purpose of this application is to provide a temperature control system and method for multiple radio frequency ablation needles, which can achieve overall heating of the ablated zone in a roughly balanced manner, avoiding the problem of excessive temperature differences between different parts of the ablated zone.

This application discloses a temperature control system for multiple radio frequency (RF) ablation needles, comprising:
N radio frequency ablation needles, each radio frequency ablation needle configured with a temperature sensor for detecting the temperature value of that radio frequency ablation needle, where N is an integer greater than 1;
A radio frequency generator, configured to provide radio frequency voltage to the N radio frequency ablation needles;
An ablation switch array, configured to make or break the electrical connection between designated radio frequency ablation needles among the N radio frequency ablation needles and the radio frequency generator;
A controller, coupled respectively to the temperature sensors on the N radio frequency ablation needles and the ablation switch array, the controller being configured to:
obtain N temperature values from the temperature sensors on the N radio frequency ablation needles,
calculate a dynamically rising current reference temperature based on the N temperature values,
determine a current upper temperature limit and a current lower temperature limit based on that current reference temperature,
control the ablation switch array, such that radio frequency ablation needles with temperature values exceeding the current upper temperature limit are disconnected from the radio frequency generator, and radio frequency ablation needles with temperature values below the current lower temperature limit are connected to the radio frequency generator.

In a preferred example, the controller is further configured to maintain an existing connection status between a radio frequency ablation needle and the radio frequency generator if the temperature value of the radio frequency ablation needle is between the current upper temperature limit and the current lower temperature limit.

In a preferred example, calculating the dynamically rising current reference temperature based on the N temperature values further includes:
selecting M temperature values from the N temperature values to calculate an average value as the current reference temperature, where N>M>1, and the M temperature values include the maximum value among the N temperature values but exclude the minimum value among the N temperature values.

In a preferred example, calculating the dynamically rising current reference temperature based on the N temperature values further includes: calculating the average value of the N temperature values as the aforementioned current reference temperature.

In a preferred example, the current upper temperature limit equals the current reference temperature plus a predetermined first temperature interval;
the current lower temperature limit equals the current reference temperature minus a predetermined second temperature interval;
where the first temperature interval and the second temperature interval are two positive numbers.

In a preferred example, the ablation switch array comprises N parallel ablation switches, respectively configured between the N radio frequency ablation needles and the radio frequency generator, for connecting or disconnecting the electrical connection between the corresponding radio frequency ablation needle and the radio frequency generator.

This application also discloses a temperature control method for multiple radio frequency ablation needles, where N radio frequency ablation needles are respectively configured with temperature sensors for detecting the temperature value of that radio frequency ablation needle, N is an integer greater than 1, and a radio frequency generator provides radio frequency voltage to the N radio frequency ablation needles through an ablation switch array; the method comprising:
obtaining N temperature values from the temperature sensors on the N radio frequency ablation needles,
calculating a dynamically rising current reference temperature based on the N temperature values, determining a current upper temperature limit and a current lower temperature limit based on that current reference temperature,
controlling the ablation switch array, such that radio frequency ablation needles with temperature values exceeding the current upper temperature limit are disconnected from the radio frequency generator, and radio frequency ablation needles with temperature values below the current lower temperature limit are connected to the radio frequency generator.

In a preferred example, the method further comprises:
controlling the ablation switch array, such that an existing connection status between a radio frequency ablation needle and the radio frequency generator is maintained when the temperature value of the radio frequency ablation needle is between the current upper temperature limit and the current lower temperature limit.

In a preferred example, calculating the dynamically rising current reference temperature based on the N temperature values further includes:
selecting M temperature values from the N temperature values to calculate an average value as the current reference temperature, where N>M>1, and the M temperature values include the maximum value among the N temperature values but exclude the minimum value among the N temperature values.

This application further discloses a non-transitory computer-readable storage medium having computer-executable instructions stored thereon, which, when executed by a processor, cause the processor to perform the foregoing method steps.

The embodiments of this application can achieve overall heating of the ablated zone in a roughly balanced manner, avoiding the problem of excessive temperature differences between different parts of the ablated zone.

Additionally, because only a single radio frequency generator is needed, compared to technical solutions requiring multiple radio frequency generators, the overall cost and complexity can be greatly reduced. By measuring the needle tip temperature through the temperature sensor built into each radio frequency ablation needle, temperature measurement is relatively accurate, conducive to precise temperature control. There is no limit to the number of electrodes that can simultaneously apply radio frequency power, enabling a larger ablation range and higher ablation efficiency.

In a preferred example, the output power of the radio frequency generator is roughly constant, with the various radio frequency ablation needles connected in parallel to the output terminal of the radio frequency generator. When some radio frequency ablation needles are disconnected due to exceeding the current upper temperature limit, the output power of the radio frequency generator is automatically redistributed among the remaining connected radio frequency ablation needles, causing the remaining connected radio frequency ablation needles to receive more radio frequency power, thereby accelerating the heating process. Consequently, this method not only ensures uniform overall heating among the radio frequency ablation needles and prevents localized overheating within the ablation zone, but it also enhances the overall heating rate for the entire ablation zone (since the radio frequency generator can always maintain maximum power output), effectively achieving adaptive power distribution. In other words, it can achieve balanced overall heating in the fastest way under conditions where the output power of the radio frequency generator remains unchanged.

The technical features disclosed in this section, the technical features disclosed in any of the following embodiments and examples, and the technical features disclosed in the drawings may be freely combined with each other to constitute various new technical solutions (these technical solutions should all be deemed as having been disclosed in the present specification), unless it is technically infeasible to combine such technical features together. For example, if feature A+B+C is disclosed in one example and feature A+B+D+E is disclosed in another example, where features C and D are equivalent technical means serving the same function, only one of which needs to be used technically and they cannot be used simultaneously, and feature E can technically be combined with feature C, then the solution A+B+C+D should not be considered as already recorded due to technical infeasibility, while the solution A+B+C+E should be considered as already recorded.

### BRIEF DESCRIPTION OF THE DRAWINGS

FIG. 1 is a schematic diagram of a temperature control system for multiple radio frequency ablation needles according to the first embodiment of this application;

FIG. 2 is a schematic diagram of the control flow executed by the controller according to an embodiment of this application;

FIG. 3 shows simulation results of temperature control for simultaneous radio frequency ablation with two needles according to an embodiment of this application;

FIG. 4 is a flow chart of a temperature control method for multiple radio frequency ablation needles according to the second embodiment of this application.

### DETAILED DESCRIPTION

In the following description, numerous technical details are set forth in order to provide a thorough understanding of the present application. However, those skilled in the art can understand that the technical solution claimed in this application can be realized without these technical details and various changes and modifications based on the following embodiments. In order to make the objectives, technical solutions, and advantages of the present application clearer, embodiments of the present application will be further described in detail below with reference to the drawings.

As mentioned earlier, technical solutions for multiple radio frequency ablation needles in the prior art may experience problems with excessive temperature differences between ablation needles. The inventor of this invention has found:
1. Large temperature differences lead to non-uniform ablation range, reducing the reliability of ablation surgery, especially for probes with lower temperatures, which may result in incomplete ablation and eventual tumor recurrence.
2. Temperature differences cannot be predicted in advance, and large differences can invalidate physician experience, manufacturer instructions, or surgical planning software.
3. Temperature control aims for precision, involving protection of sensitive tissues (such as liver capsule, gallbladder, and other normal tissues) and precise control of tumor edge temperature (which helps stimulate immune response), large temperature differences contradict the general requirement for precision.

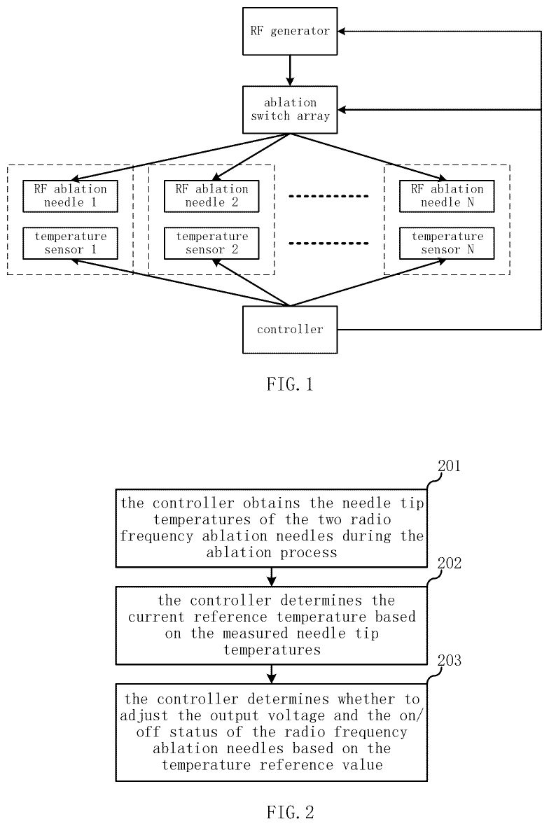

The first embodiment of this application relates to a temperature control system for multiple radio frequency ablation needles to solve the above problems. The structure of the system is shown in FIG. 1, and the system includes:
N radio frequency ablation needles, each radio frequency ablation needle configured with a temperature sensor for detecting the temperature value of that radio frequency ablation needle, where N is an integer greater than 1. Optionally, in an embodiment, each radio frequency ablation needle has a temperature sensor integrated at its tip, enabling integration of ablation and temperature measurement, and the temperature sensor can realize real-time measurement of the temperature at the ablation end of the radio frequency ablation needle. Each radio frequency ablation needle can form an ablation zone, and the temperature value measured by the temperature sensor is the central temperature of the ablation zone formed by that radio frequency ablation needle. The temperature sensor can be a thermocouple, temperature measuring optical fiber, etc. The temperature sensor can be integrated at any position inside or outside the radio frequency ablation needle (the needle tip is a preferred position). Each radio frequency ablation needle can integrate one or more temperature sensors.

A radio frequency generator, configured to provide radio frequency voltage to the N radio frequency ablation needles. Optionally, in an embodiment, the radio frequency generator can generate high-frequency alternating voltage of around 460 KHz, forming a closed circuit with the negative plate, creating high-frequency alternating current that flows through human tissue to produce thermal effects.

An ablation switch array, configured to make or break the electrical connection between designated radio frequency ablation needles among the N radio frequency ablation needles and the radio frequency generator. The ablation switch array can independently control the on-off status of the radio frequency voltage applied to each radio frequency ablation needle. The ablation switch array includes multiple ablation switches. Ablation switches can be implemented in various ways, such as switching transistors, relays, thyristors, etc.

A controller, coupled respectively to the temperature sensors on the N radio frequency ablation needles and the ablation switch array, the controller being configured to: obtain N temperature values from the temperature sensors on the N radio frequency ablation needles; calculate a dynamically rising current reference temperature based on the N temperature values, determine a current upper temperature limit and a current lower temperature limit based on that current reference temperature; control the ablation switch array, such that radio frequency ablation needles with temperature values exceeding the current upper temperature limit are disconnected from the radio frequency generator, and radio frequency ablation needles with temperature values below the current lower temperature limit are connected to the radio frequency generator.

Optionally, in an embodiment, the controller is further configured to maintain the existing connection status between radio frequency ablation needles (with temperature values between the current upper temperature limit and the current lower temperature limit) and the radio frequency generator. That is to say, when the current temperature value of a radio frequency ablation needle is between the current upper temperature limit and the current lower temperature limit, if the radio frequency ablation needle was originally connected to the radio frequency generator, it continues to maintain the connected status; if it was originally disconnected from the radio frequency generator, it continues to maintain the disconnected status. This can avoid frequent changes in the connection status between the radio frequency ablation needle and the radio frequency generator.

Optionally, in an embodiment, the output power of the radio frequency generator is roughly constant, with the various radio frequency ablation needles connected in parallel to the output terminal of the radio frequency generator. When some radio frequency ablation needles are disconnected due to exceeding the current upper temperature limit, the remaining connected radio frequency ablation needles will receive more radio frequency power than before, thereby accelerating the heating process. In this way, not only is uniform overall heating among the radio frequency ablation needles ensured, preventing local overheating of the ablated zone, but the overall heating speed of the ablated zone is also enhanced, creating an adaptive power distribution effect.

Optionally, in an embodiment, the output voltage of the radio frequency generator is roughly constant, and regardless of the number of radio frequency ablation needles currently connected to the radio frequency generator, the power on each radio frequency ablation needle in the connected status is roughly constant.

Optionally, in an embodiment, calculating the dynamically rising current reference temperature based on the N temperature values further includes: calculating the average value of the N temperature values as the aforementioned current reference temperature. In another embodiment, it may not necessarily be an arithmetic mean, but a weighted average, with weights that can be predetermined. In an alternative embodiment, an average value may also be calculated based solely on the temperatures of the respective radio frequency ablation needles engaged with the radio frequency generator.

Optionally, in an embodiment, calculating the dynamically rising current reference temperature based on the N temperature values further includes: selecting M temperature values from the N temperature values to calculate an average value as the current reference temperature, where N>M>1, and the M temperature values include the maximum value among the N temperature values but exclude the minimum value among the N temperature values. For example, if N=3 and M=2, the average of the 2 higher temperature values can be calculated as the current reference temperature. It should be noted that in this embodiment, the highest temperature value needs to be included, while the lowest temperature value needs to be excluded, which can prevent tissue carbonization problems due to excessively high temperatures in the radio frequency ablation needle with the highest temperature. For example, if the set temperature is 97° C., and 3 probes are stable at 100° C., 97° C., and 94° C. respectively, if the highest and lowest are removed, the average value is 97° C., and the temperature differences of all 3 needles are within the upper and lower thresholds, power is maintained. However, after a period of time, the tissue around the first probe becomes dehydrated and carbonized, making continued treatment impossible. If the highest is not removed, the reference temperature is higher than the set value, power is reduced, and there is no carbonization risk. This approach differs from the conventional means of achieving average, which typically removes both the highest and lowest values simultaneously. The current reference temperature is usually also compared with a target temperature, continuously increasing the temperature until the current reference temperature reaches the target temperature.

Optionally, in an embodiment, the current upper temperature limit equals the current reference temperature plus a predetermined first temperature interval. The current lower temperature limit equals the current reference temperature minus a predetermined second temperature interval. Here, the first temperature interval and the second temperature interval are two positive numbers.

Optionally, in an embodiment, the first temperature interval may equal the second temperature interval.

Optionally, in an embodiment, the first temperature interval may be not equal to the second temperature interval.

Optionally, in an embodiment, the current upper temperature limit may equal the current lower temperature limit, for example, both the current upper temperature limit and the current lower temperature limit can equal the current reference temperature. The advantage of doing this may be that there is no need to calculate the current upper temperature limit and the current lower temperature limit, but the disadvantage is that some ablation switches may frequently switch between disconnected and connected status.

Optionally, in an embodiment, the ablation switch array comprises N parallel ablation switches, respectively configured between the N radio frequency ablation needles and the radio frequency generator, for connecting or disconnecting the electrical connection between the corresponding radio frequency ablation needle and the radio frequency generator. In other embodiments of this application, there can also be more or fewer ablation switches, and the connection method is not limited to each ablation switch being connected between one radio frequency ablation needle and the radio frequency generator. As long as independent control of the connection between each radio frequency ablation needle and the radio frequency generator can be achieved, it is acceptable.

Optionally, in an embodiment, in addition to adjusting the connection status between various radio frequency ablation needles and the radio frequency generator based on the current reference temperature, the output voltage of the radio frequency generator may also be adjusted based on the current reference temperature.

In some embodiments of this application, the controller can be implemented in various ways, such as a Central Processing Unit (CPU), Graphic Processing Unit (GPU), Digital Signal Processor (DSP), Microcontroller Unit (MCU), Neural Processing Unit (NPU), Application Specific Integrated Circuit (ASIC), Field Programmable Gate Array (FPGA), or other programmable logic devices. It can be implemented entirely by hardware, or through a combination of software and hardware.

To better understand the technical solution of this application, the following explains it in conjunction with a specific example of dual-needle radio frequency ablation. The details listed in this example are primarily for understanding and do not limit the scope of protection of this application.

There are two radio frequency ablation needles, each connected to the same radio frequency generator through two ablation switches. Each radio frequency ablation needle integrates a thermocouple as a temperature sensor.

The controller is a processor, and the executable code running on it performs the following operations (as shown in FIG. 2):

In step 201, the controller obtains the needle tip temperatures of the two radio frequency ablation needles during the ablation process. The needle tip temperature may be measured by the thermocouples integrated into the needle tips of the radio frequency ablation needles and stored in a storage medium (such as memory), and read in real time by the program.

Then, in step 202, the controller determines the current reference temperature based on the measured needle tip temperatures.

In this embodiment, there is a relatively obvious difference in the blood perfusion rate level of the tissue at the locations of the two radio frequency ablation needles, so the temperature rise rates of the two radio frequency ablation needles will have considerable differences. To make the temperatures of both radio frequency ablation needles as stable and controllable as possible, the average value AVE of the needle tip temperatures of the two radio frequency ablation needles is selected as the reference temperature. If the number of radio frequency ablation needles is greater than or equal to 3, the lowest needle tip temperature can be removed, and the average value AVE of the other needle tip temperatures is used as the reference temperature, thereby avoiding the influence of radio frequency ablation needles around special tissue structures (such as blood vessels) on the temperature control of radio frequency ablation needles in other normal positions.

Then, in step 203, the controller determines whether to adjust the output voltage and the on/off status of the radio frequency ablation needles based on the temperature reference value.

In this embodiment, the preset temperature is TARG° C., and the current reference temperature obtained in step 202 is AVE° C. Based on the difference between them, the following control equation is used to adjust the output voltage of the radio frequency generator:

$${V}_{pi} = {k}_{p} * {c}_{ht} * error + {k}_{i} * {\int }_{0}^{t} error dt$$

$$error = TARG - AVE$$

where Vₚᵢ is the output voltage of the radio frequency generator, TARG is the set temperature value, AVE is the current reference temperature, kₚ and kᵢ are the preset proportional coefficient and integral coefficient respectively, error is the difference between the temperature set value and the current reference temperature, t represents time, and cₕₜ is the proportional coefficient correction parameter related to the highest probe temperature:

$${c}_{ht} = \frac{{T}_{danger} - {T}_{max}}{{T}_{danger} - P}$$

where Tₘₐₓ is the highest value of the needle tip temperature values of the radio frequency ablation needles currently in the connected status with the radio frequency generator, T_{danger} is the preset dangerous upper temperature limit related to the target ablation tissue, and P is an empirical parameter (for example, P may be 75). Introducing cₕₜ may accelerate the heating rate in low-temperature status and suppress the proportional coefficient when approaching dangerous temperatures, increasing safety.

In addition, thresholds th1 and th2 may be set based on the current reference temperature (for example, th1=current reference temperature+C, th2=current reference temperature−C, where C is a preset positive number). When the temperature of any needle tip exceeds the reference temperature th1, the switch of that radio frequency ablation needle is disconnected, and the temperature of the disconnected probe is not included in the calculation of the average needle tip temperature AVE. When the temperature of any needle tip is below the reference temperature th2, the switch of that radio frequency ablation needle is turned on. Therefore, the switch thresholds of the radio frequency ablation needles are dynamically changing, making the heating rate of each probe as consistent as possible, reducing overshoot and oscillation phenomena.

According to the above control equation, the output voltage of the radio frequency generator is adjusted. During the heating process, the role of the proportional term can be gradually weakened (for example, when the highest temperature exceeds 75° C., Cₕₜ in the equation will be less than 1), and the power adjustment relies more on the integral term, making the temperature rise more stable, reducing or even eliminating the overshoot of the highest temperature.

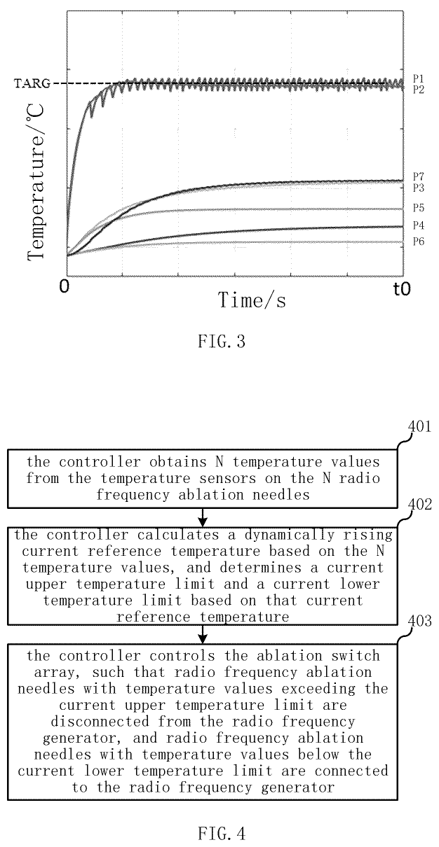

As shown in FIG. 3, the result of temperature control simulation for simultaneous dual-needle radio frequency ablation at t0 seconds, where P1 to P7 each correspond to a temperature curve over time, and each curve is marked according to the order from high to low temperature at the t0 moment. P1 and P2 are the two needle tips, P3 and P5 are points 5 mm away from the outer sides of the walls of the two radio frequency ablation needles, P4 and P6 are points 10 mm away from the outer sides of the walls of the two radio frequency ablation needles, and P7 is the point between the two needles. The needle tip temperatures of both radio frequency ablation needles are well maintained near the target temperature TARG° C. and are relatively stable. This demonstrates that this system and temperature control method can achieve precise temperature control in simultaneous dual-needle radio frequency ablation.

The second embodiment of this application relates to a temperature control method for multiple radio frequency ablation needles, with the flow shown in FIG. 4. N radio frequency ablation needles are respectively configured with temperature sensors for detecting the needle tip temperature of that radio frequency ablation needle, N is an integer greater than 1, and a radio frequency generator provides radio frequency voltage to the N radio frequency ablation needles through an ablation switch array. The method includes the following steps:

Step 401, the controller obtains N temperature values from the temperature sensors on the N radio frequency ablation needles.

Then, in step 402, the controller calculates a dynamically rising current reference temperature based on the N temperature values, and determines a current upper temperature limit and a current lower temperature limit based on that current reference temperature.

Then, in step 403, the controller controls the ablation switch array, such that radio frequency ablation needles with temperature values exceeding the current upper temperature limit are disconnected from the radio frequency generator, and radio frequency ablation needles with temperature values below the current lower temperature limit are connected to the radio frequency generator. Optionally, in an embodiment, the ablation switch array can also be controlled such that radio frequency ablation needles with temperature values between the current upper temperature limit and the current lower temperature limit maintain their existing connection status with the radio frequency generator.

Optionally, in an embodiment, calculating the dynamically rising current reference temperature in step 404 based on the N temperature values can further include: selecting M temperature values from the N temperature values to calculate an average value as the current reference temperature, where N>M>1, and the M temperature values include the maximum value among the N temperature values but exclude the minimum value among the N temperature values.

The first aspect is a method implementation corresponding to the second aspect, and the technical details in the first aspect can be applied to the second aspect, and the technical details in the second aspect can also be applied to the first aspect.

Furthermore, the embodiments of this application also provide a non-transitory computer-readable storage medium storing computer-executable instructions that, when executed by a processor, implement the steps of the various method embodiments of this application. The computer-readable storage media include permanent and non-permanent, removable and non-removable media that can store information by any method or technology. Information can be computer-readable instructions, data structures, program modules, or other data. Examples of the computer-readable storage media include, but are not limited to, Phase-Change Random Access Memory (PRAM), Static Random Access Memory (SRAM), Dynamic Random Access Memory (DRAM), other types of Random Access Memory (RAM), Read-Only Memory (ROM), Electrically Erasable Programmable Read-Only Memory (EEPROM), flash memory or other memory technologies, Compact Disc Read-Only Memory (CD-ROM), Digital Versatile Disc (DVD) or other optical storage, magnetic cassettes, magnetic disk storage or other magnetic storage devices, or any other non-transmission medium that can be used to store information accessible by a computing device. As defined herein, the computer-readable storage media exclude transitory media, such as modulated data signals and carriers.

Furthermore, the embodiments of this application also provide a temperature control system for multiple radio frequency ablation needles, which includes a memory for storing computer-executable instructions, and a processor; the processor is used to implement the steps in the above method embodiments when executing the computer-executable instructions in the memory. The processor may be a Central Processing Unit (CPU), or it may be other general processors, Digital Signal Processors (DSP), Application Specific Integrated Circuits (ASIC), etc. The aforementioned memory may be Read-Only Memory (ROM), Random Access Memory (RAM), flash memory, hard disk, or solid-status disk, etc. The steps of the method disclosed in the embodiments of the present invention may be directly embodied by a hardware processor, or may be implemented by a combination of hardware and software modules in a processor.

It should be noted that, in this application, relational terms such as “first”, “second”, etc. are used solely to distinguish one entity or operation from another without necessarily requiring or implying any actual relationship or order between the entities or operations. Moreover, the term “comprise”, “include”, or any other variants thereof are intended to cover a non-exclusive inclusion, such that a process, method, article, or apparatus that comprises a list of elements not only comprises those elements, but may also comprise other elements not expressly listed or inherent to such a process, method, article, or apparatus. Without further limitations, an element defined by the statement “comprising a” does not exclude the presence of additional identical elements in the process, method, article, or apparatus that comprises the element. In this application, if it is mentioned that an action is performed based on a certain element, it means performing that action based on at least that element, which includes two situations: (1) the behavior is performed only according to that element, and (2) the behavior is performed according to that element and other elements. The expressions ‘multiple’ and ‘a plurality of’ are defined to mean two or more than two.

The sequence numbers used in describing the steps of a method themselves do not impose any limitations on the order of these steps. For example, a step with a larger sequence number does not necessarily have to be executed after a step with a smaller sequence number; it can also be executed first with the larger sequence number and then with the smaller sequence number, or they can be executed in parallel, as long as this execution order is reasonable for those skilled in the art. Also, steps with consecutive sequence numbers (such as step 201, step 202, step 203, etc.) do not limit other steps from being executed in between; for example, there can be other steps between step 201 and step 202.

The specification includes combinations of the various embodiments described herein. Separate references to embodiments (e.g. “an embodiment” or “some embodiments” or “preferred embodiments”) do not necessarily refer to the same embodiment; however, these embodiments are not mutually exclusive unless indicated as such or as will be apparent to those skilled in the art. It should be noted that the word “or” is used in this specification in a non-exclusive sense unless the context expressly indicates or requires otherwise.

All documents mentioned in this specification are deemed as included in the disclosure of this application in their entirety so that they may serve as a basis for amendment if necessary. In addition, it shall be understood that the foregoing are merely better examples of the specification and are not intended to limit the scope of protection of the patent. Any modification, equivalent replacement, improvement, etc. within the spirit and principles of one or more embodiments of this specification shall be included in the scope of protection of such one or more embodiments of this specification.

In some cases, the actions or steps described in the claims may be executed in an order different from that shown in the embodiments and still achieve the desired results. Additionally, the processes depicted in the drawings do not necessarily require the specific order or sequential order shown to achieve the desired results. In certain embodiments, multi-tasking and parallel processing may also be possible or advantageous.

1. A temperature control system for multiple radio frequency ablation needles, comprising:
N radio frequency ablation needles, each radio frequency ablation needle being configured with a temperature sensor for detecting a temperature value of the radio frequency ablation needle, wherein N is an integer greater than 1;
a radio frequency generator, providing radio frequency voltage to the N radio frequency ablation needles;
an ablation switch array being operable to make or break an electrical connection between designated radio frequency ablation needles of the N radio frequency ablation needles and the radio frequency generator;
a controller, coupled respectively to the temperature sensors on the N radio frequency ablation needles and the ablation switch array, the controller being configured to:
obtain N temperature values from the temperature sensors on the N radio frequency ablation needles,
calculate a dynamical current reference temperature based on the N temperature values,
determine a current upper temperature limit and a current lower temperature limit based on the current reference temperature,
control the ablation switch array, such that radio frequency ablation needles with temperature values exceeding the current upper temperature limit are disconnected from the radio frequency generator, and radio frequency ablation needles with temperature values below the current lower temperature limit are connected to the radio frequency generator.

2. The temperature control system for multiple radio frequency ablation needles of claim 1, wherein
the controller is further configured to maintain an existing connection status between a radio frequency ablation needle and the radio frequency generator if the temperature value of the radio frequency ablation needle is between the current upper temperature limit and the current lower temperature limit.

3. The temperature control system for multiple radio frequency ablation needles of claim 1, wherein calculating the dynamical current reference temperature based on the N temperature values, further comprises:
selecting M temperature values from the N temperature values to calculate an average value as the current reference temperature, wherein N>M>1, and the M temperature values include the maximum value among the N temperature values but exclude the minimum value among the N temperature values.

4. The temperature control system for multiple radio frequency ablation needles of claim 1, wherein
calculating the dynamical current reference temperature based on the N temperature values, further comprises: calculating an average value of the N temperature values as the current reference temperature.

5. The temperature control system for multiple radio frequency ablation needles of claim 1, wherein
the current upper temperature limit equals the current reference temperature plus a predetermined first temperature interval;
the current lower temperature limit equals the current reference temperature minus a predetermined second temperature interval;
wherein, the first temperature interval and the second temperature interval are two positive numbers.

6. The temperature control system for multiple radio frequency ablation needles of claim 1, wherein the ablation switch array comprises N parallel ablation switches, respectively connected between the N radio frequency ablation needles and the radio frequency generator, for connecting or disconnecting electrical connection between corresponding radio frequency ablation needles and the radio frequency generator.

7. A temperature control method for multiple radio frequency ablation needles, wherein N radio frequency ablation needles are respectively configured with temperature sensors for detecting temperature values of the radio frequency ablation needles, N is an integer greater than 1, and a radio frequency generator provides radio frequency voltage to the N radio frequency ablation needles through an ablation switch array; the method comprising:
obtaining N temperature values from the temperature sensors on the N radio frequency ablation needles,
calculating a dynamical current reference temperature based on the N temperature values,
determining a current upper temperature limit and a current lower temperature limit based on the current reference temperature,
controlling the ablation switch array, such that radio frequency ablation needles with temperature values exceeding the current upper temperature limit are disconnected from the radio frequency generator, and radio frequency ablation needles with temperature values below the current lower temperature limit are connected to the radio frequency generator.

8. The temperature control method for multiple radio frequency ablation needles of claim 7, further comprising:
controlling the ablation switch array, such that an existing connection status between a radio frequency ablation needle and the radio frequency generator is maintained when the temperature value of the radio frequency ablation needle is between the current upper temperature limit and the current lower temperature limit.

9. The temperature control method for multiple radio frequency ablation needles of claim 7, wherein calculating the dynamical current reference temperature based on the N temperature values, further comprises:
selecting M temperature values from the N temperature values to calculate an average value as the current reference temperature, wherein N>M>1, and the M temperature values include the maximum value among the N temperature values but exclude the minimum value among the N temperature values.

10. The temperature control method for multiple radio frequency ablation needles of claim 7, further comprising, adjusting the output voltage Vₚᵢ of the radio frequency generator according to the following control equation,
$${V}_{pi} = {k}_{p} * {c}_{ht} * error + {k}_{i} * {\int }_{0}^{t} error dt$$
$$error = TARG - AVE$$
$${c}_{ht} = \frac{{T}_{danger} - {T}_{max}}{{T}_{danger} - P}$$
wherein, TARG is a preset target temperature value, AVE is the current reference temperature, kₚ and kᵢ are preset proportional coefficient and integral coefficient respectively, t represents time, cₕₜ is a proportional coefficient correction parameter related to the highest probe temperature, Tₘₐₓ is the highest temperature value among the needle tip temperature values of the radio frequency ablation needles currently connected to the radio frequency generator, T_{danger} is a preset dangerous upper temperature limit related to the target ablation tissue, P is a preset empirical parameter.
